(19) United States
(12) Patent Application Publication
(10) Pub. No.: US 2020/0337509 A1
(43) Pub. Date: Oct. 29, 2020

(54) SMART TOILET SAFE IN USE

(71) Applicant: Guangdong Tak Long Times Smart Home Co.,Ltd, Guangdong (CN)

(72) Inventor: Kevin Ho, Guangdong (CN)

(73) Assignee: Guangdong Tak Long Times Smart Home Co.,Ltd, Guangdong (CN)

(21) Appl. No.: 16/553,183

(22) Filed: Aug. 28, 2019

(30) Foreign Application Priority Data
Apr. 28, 2019 (CN) 201920603113.9

(51) Int. Cl. A47K 13/30; A47K 13/10

(52) U.S. Cl. CPC A47K 13/302; A61L 2/07; A47K 13/10

(57) ABSTRACT

The present disclosure discloses a safe smart toilet, which includes a toilet seat, a rear seat, a seat ring, a toilet cover, a steam generating mechanism with a fumigating and sterilizing function, an automatic opening and closing mechanism and a main control circuit board. The steam generating mechanism includes a steam generator and a radar sensor. The steam generator is arranged in the toilet seat through a slide rail mechanism, and the steam generator is provided with a water inlet and a sewage outlet for cleaning a heater. The automatic opening and closing mechanism includes a turning motor, a flip motor, a turning sensor, and a flip sensor. The smart toilet of the present disclosure uses steam to perform high-temperature sterilization on the toilet seat and the seat ring to improve the cleanliness thereof.

### CROSS-REFERENCE TO RELATED APPLICATION

This application claims the priority benefits of Chinese application serial no. 201920603113.9, filed on Apr. 28, 2018. The entirety of the above-mentioned patent application is hereby incorporated by reference herein and made a part of specification.

### BACKGROUND

### Technical Field

The present disclosure relates to the technical field of toilets, in particular to a smart toilet which is safe in use.

### Description of Related Art

With the continuous development of science and technology and the continuous advancement of society, people's life is becoming more and more intelligent, for example, the invention of smart toilets. Known smart toilet can realize functions such as automatic cleaning, automatic flushing, automatic flipping, etc. Generally, in automatic cleaning, the smart toilet is fumigated and disinfected by steam, and the steam ejected is high-temperature steam. If a user's body is close to the steam, the user's body is likely to get burned and unnecessary safety issues will arise, in that case, the safety performance is low. Besides, for the steam generator that generates steam, since scale will be generated in the steam generator after the water is heated for a long period of time, if the scale is not removed timely, not only that the heat conversion efficiency will be reduced, but also the energy consumption will be increased. Moreover, the heater is a humid environment containing water for a long period of time, bacteria is likely to be accumulated, which will cause health and hygiene issues.

In view of the above, the above issues are technical problems to be solved by practitioners of the field.

### SUMMARY

The main purpose of the present disclosure is to provide a smart toilet which is safe in use, and aims to realize a smart toilet with reasonable structural design, clean and hygienic, high safety performance and high heating efficiency.

The present disclosure provides a smart toilet safe in use. The smart toilet includes a toilet seat, a rear seat, a seat ring and a toilet cover hinged on the rear seat through a rotating shaft. The safe smart toilet further includes a steam generating mechanism which is disposed in the toilet seat and can spray steam with fumigating and disinfecting functions toward the toilet seat, an automatic opening and closing mechanism disposed in the rear seat and driving the seat ring and the toilet cover to open automatically, a main control circuit board which is arranged in the rear seat and controls operation of the steam generating mechanism and the automatic opening and closing mechanism. The steam generating mechanism includes a steam generator and a radar sensor connected to the main control circuit board. The steam generator is arranged in the toilet seat through the slide rail mechanism, and the toilet seat is provided with a cleaning hole that corresponds in position to the slide rail mechanism and that allows the steam generator to slide out. The automatic opening and closing mechanism includes a turning motor, a flip motor, a turning sensor and a flip sensor that are disposed in the toilet cover and connected to the main control circuit board.

Preferably, the steam generator is provided with a water inlet connected to a water pump, a sewage outlet for cleaning the heater and a steam outlet. The steam generator includes a housing, a heat insulation shell, a heater cover, a heater, a temperature controller, and a sewage outlet cover. The temperature controller is connected to the main control circuit board.

Preferably, the slide rail mechanism includes an inner slide rail fixed in the toilet seat, an outer slide rail fastened on the inner slide rail and sliding in a left-right direction along the inner slide rail. The steam generator is fixed on the outer slide rail.

Preferably, the toilet cover is provided with an inner cavity, and a lower end surface of the inner cavity is provided with a plurality of steam injecting holes at a position corresponding to a position of the seat ring, and the inner cavity communicates with the steam outlet through the conduit.

Preferably, the turning sensor and the flip sensor are configured as a microwave sensor head or an infrared sensor head.

Preferably, the rear seat is provided with a control panel connected to the main control circuit board, and the control panel is provided with a plurality of control buttons and indicator lights.

Preferably, the cleaning hole is provided with a cleaning cover.

The present disclosure uses the smart toilet safe in use, adopts steam to perform high-temperature disinfection and sterilization on the toilet seat and the seat ring, thereby improving the cleanliness of the smart toilet and effectively killing the bacteria to clean the toilet, so as to prevent the occurrence and propagation of the disease. Moreover, the main control circuit board is connected to a radar sensor. When the steam sterilization button is pressed, the radar sensor starts operating and detects whether there is anyone moving within 6 meters. If human activity is detected, the main control circuit board controls the steam generator not to operate according to the signal detected by the radar sensor, thereby preventing the generated high-temperature steam from burning a user's body. When the user leaves a detection range, the main control circuit board controls the steam generator to operate and perform fumigation and sterilization on the smart toilet. If the steam generator is working and performing fumigation and sterilization on the toilet seat while someone enters a sensing range of the radar sensor, the main control circuit board controls the steam generator to stop working according to the signal detected by the radar sensor and to stop injecting the steam, thereby effectively improving the safety performance and intelligent use of the smart toilet. The structure is simple and the design is reasonable. In addition, the steam generator can slide out from the toilet seat through the slide rail mechanism for ease of cleaning. The steam generator is provided with a water inlet connected to the water pump and the sewage outlet for cleaning the heater, and the heater is brushed through the sewage outlet. Furthermore, the water pump is used to spray water and clean the filth in the heater, the scale is discharged from the sewage outlet, so that the scale can be removed timely and the heating efficiency can be enhanced, and the steam generator can be kept clean and hygienic for a long time, so as to further improve the sanitary condition of the smart toilet.

### BRIEF DESCRIPTION OF THE DRAWINGS

FIG. 1 is a schematic perspective view of an embodiment of a smart toilet safe in use according to the present disclosure, wherein the smart toilet is in a state with a closed cover.

FIG. 2 is a second schematic perspective view of the embodiment of the smart toilet safe in use according to the present disclosure, wherein the smart toilet is in a state with an opened cover.

FIG. 3 is a third schematic perspective view of the embodiment of the smart toilet safe in use according to the present disclosure, wherein the smart toilet is in the state with the opened cover, and a radar sensor and a main control circuit board are shown.

FIG. 4 is a schematic perspective and enlarged view showing a steam generator in the embodiment of the smart toilet safe in use of the present disclosure.

FIG. 5 is a schematic exploded and enlarged view showing a steam generator in the embodiment of the smart toilet safe in use of the present disclosure.

The realization, functional features and advantages of the purpose of the present disclosure will be further described with reference to the accompanying drawings.

### DESCRIPTION OF THE EMBODIMENTS

It should be understood that the specific embodiments described herein are merely illustrative of the disclosure and are not intended to limit the disclosure.

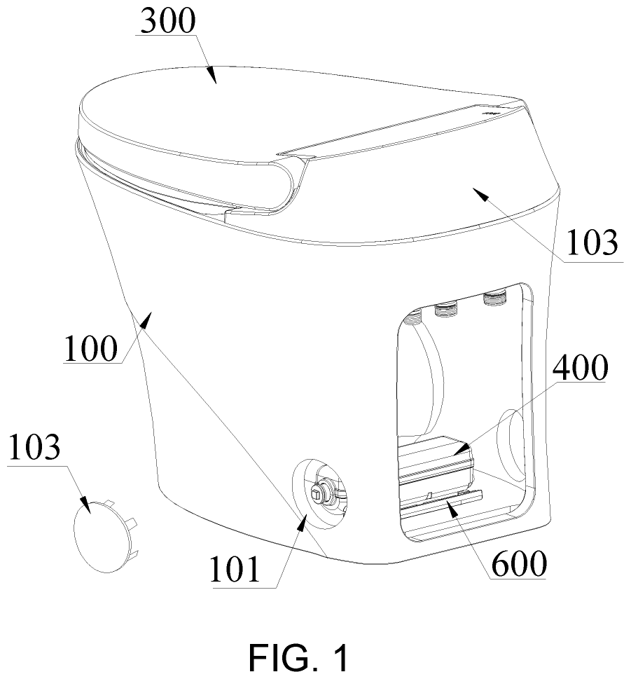

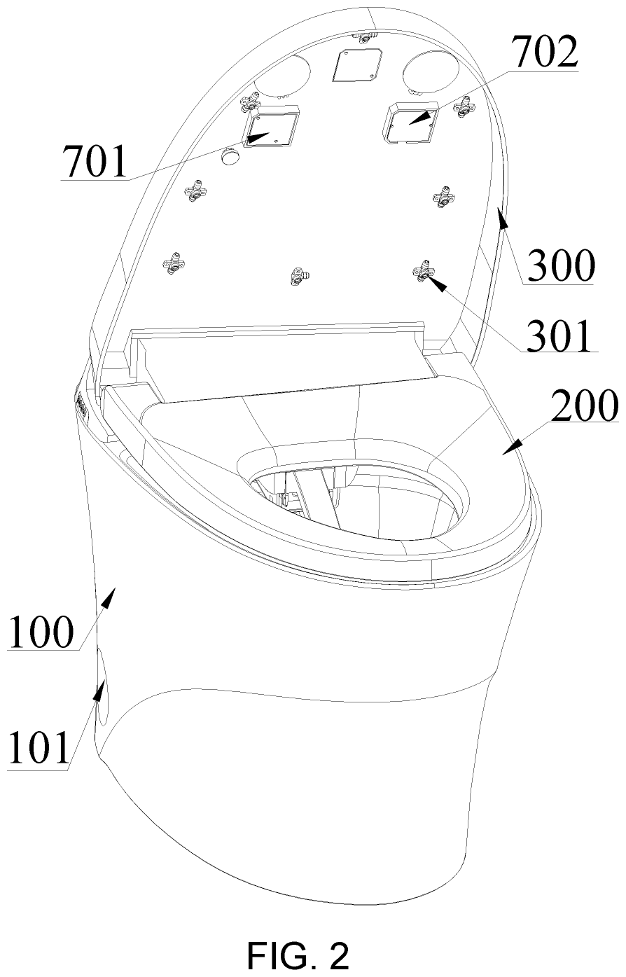

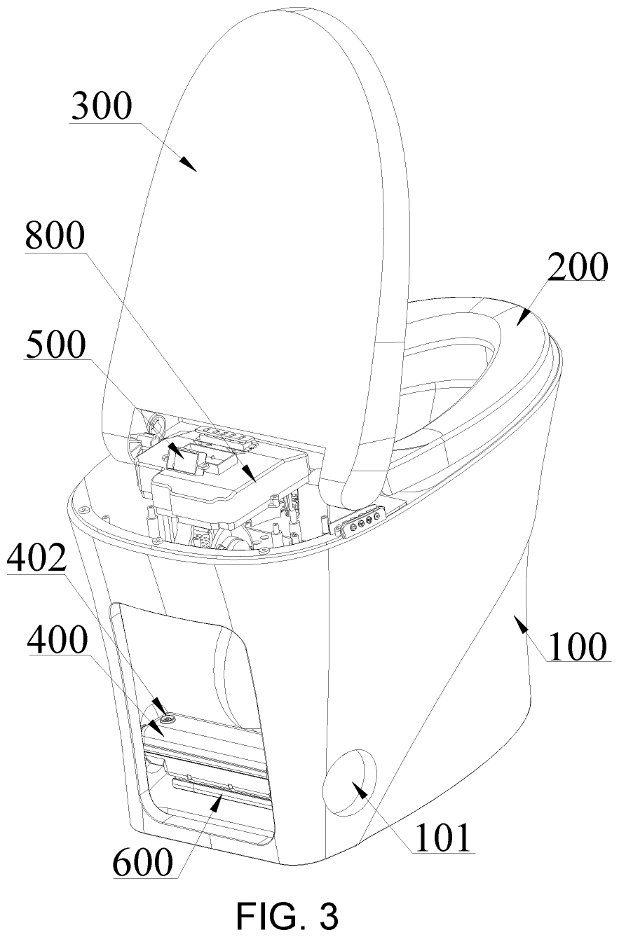

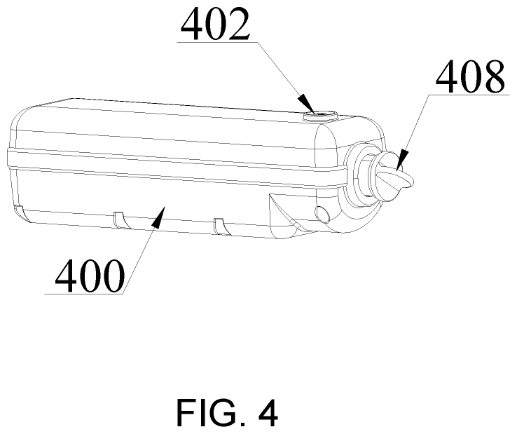

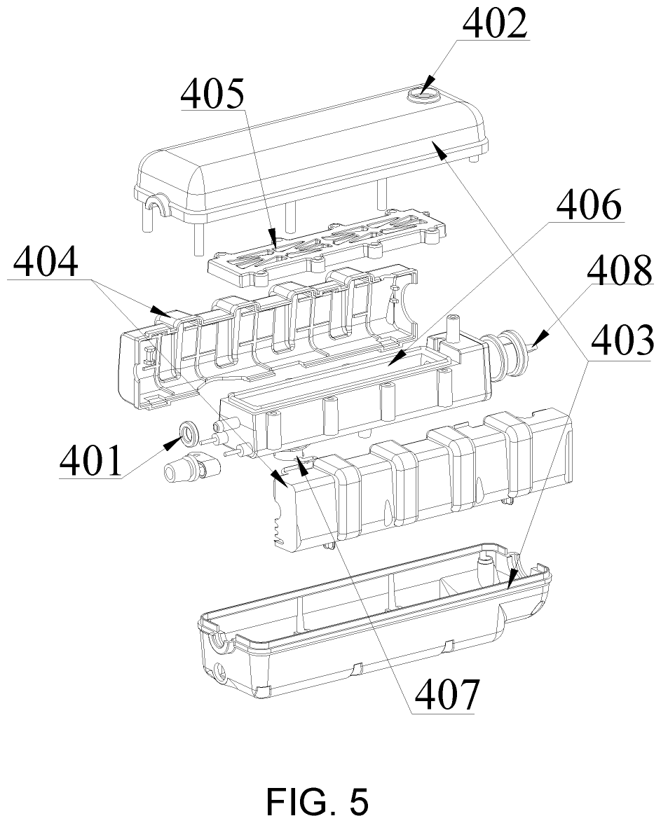

Referring to FIG. 1 to FIG. 5, an embodiment of a smart toilet safe in use of the present disclosure is provided. The smart toilet includes a toilet seat 100, a rear seat 102, a seat ring 200 and a toilet cover 300 hinged on the rear seat 102 through a rotating shaft. The present disclosure further includes a steam generating mechanism and an automatic opening and closing mechanism. The steam generating mechanism is arranged in the toilet seat 100 and can spray steam with a fumigation and sterilizing function toward the toilet seat 100. The automatic opening and closing mechanism is arranged in the rear seat 102 and drives the seat ring 200 and toilet cover 300 open automatically. The smart toilet further includes a main control circuit board 800 disposed in the rear seat 102 and controlling the operation of the steam generating mechanism and the automatic opening and closing mechanism.

The steam generating mechanism includes a steam generator 400. The steam generator 400 is provided with a water inlet 401 connected to a water pump, a sewage outlet for cleaning the heater 406 and a steam outlet 402. The sewage outlet is provided with a sewage outlet cover 408. The steam generator 400 includes a housing 403, a heat insulation shell 404, a heater cover 405, a heater 406, and a temperature controller 407, wherein the temperature controller 407 is connected to the main control circuit board 800. The toilet cover 300 is provided with an inner cavity, and a lower end surface of the inner cavity is provided with a plurality of steam injecting holes 301 at a position corresponding to a position of the seat ring 200, and the inner cavity communicates with the steam outlet 402 through a conduit.

When the steam generating mechanism is working, the water pump pumps water into the steam generator 400. The heater 406 works and converts the water into steam. The steam enters the inner cavity of the toilet cover 300 through a conduit, and the high-temperature and high-pressure steam is sprayed toward the toilet seat 100 and the toilet ring 200 from the steam injecting hole 301 to achieve the purpose of fumigation, disinfection and sterilization. Meanwhile, the heater 406 is provided with the temperature controller 407 connected to the main control circuit board 800. The temperature controller 407 controls the highest temperature of the heater 406 to be within a certain range, thereby preventing the steam temperature from being too high and damaging the steam generator 400 or the seat ring 200. Moreover, it is possible to prevent dry burning through intelligent temperature control. That is, if dry burning occurs, a temperature of the heater 406 is excessively high, and a temperature value monitored by the temperature controller 407 exceeds a set range, then a signal is sent to the main control circuit board 800. After the main control circuit board 800 analyzes the received signal, the heater 406 is controlled to be powered off and stopped to ensure the normal operation of the steam generator 400 in a long period of time, thereby preventing damage caused by high temperature and prolonging service life thereof.

The steam generating mechanism further includes a radar sensor 500 connected to the main control circuit board 800. The radar sensor 500 can detect whether there is anyone moving within a certain range around the smart toilet, thereby sending a detection signal to the main control circuit board 800. The main control circuit board 800 controls the steam generator 400 to work according to the signal detected by the radar sensor 500 to improve the safety performance of the smart toilet. When the steam generating mechanism is activated, the radar sensor 500 operates and detects whether there is anyone moving within 6 meters. If human activity is detected, the main control circuit board 800 controls the steam generator 400 not to operate according to the signal detected by the radar sensor 500, so as to prevent the generated high-temperature steam from burning a user's body. When the user's body leaves a detection range, the main control circuit board 800 controls the steam generator 400 to operate and perform fumigation and sterilization on the smart toilet. If the steam generator 400 is working and performing fumigation and sterilization on the toilet seat 100 and the seat ring 200 while someone enters the sensing range of the radar sensor 500, the main control circuit board 800 controls the steam generator 400 to stop working according to the signal detected by the radar sensor 500 and to stop injecting the steam, thereby effectively improving safety performance in use and intelligent use of the smart toilet and preventing children from being accidentally burned by the high-temperature steam when moving around the toilet seat 100. The structure is simple with reasonable design and high utility.

The steam generator 400 of the present disclosure is disposed in the toilet seat 100 through the slide rail mechanism 600. The toilet seat 100 is provided with a cleaning hole 101 corresponding in position to the slide rail mechanism 600 for the steam generator 400 to slide out. The cleaning hole 101 is provided with a cleaning cover 103, and the cleaning cover 103 has a shielding function to prevent dust and smudge from entering the toilet seat 100 through the cleaning hole 101. The slide rail mechanism 600 includes an inner slide rail fixed in the toilet seat 100, an outer slide rail that is fastened on the inner slide rail and slides in a left-right direction along the inner slide rail. The steam generator 400 is fixed on the outer slide rail, and the outer slide rail and the inner slide rail can slide relative to each other, such that the steam generator 400 can slide out of the toilet seat 100 from the inside of the toilet seat 100 to facilitate cleaning of the steam generator 400. During the cleaning operation, the cleaning cover 103 is removed, the steam generator 400 is slid and pulled out from the toilet seat 100 through the cleaning hole 101, the sewage cover is unscrewed, and a cleaning brush is inserted into the heater 406 through the sewage outlet for brushing. After the brushing work is completed, the scale and sewage in the steam generator 400 is flushed out from the sewage outlet by using the water pump, and finally the scale and sewage is discharged and the sewage cover is placed back. The steam generator 400 is smoothly slid into the toilet seat 100, and the cleaning cover 103 is placed back, and the whole process is completed. By using the slide rail mechanism 600, the steam generator 400 in the toilet seat 100 can be cleaned and maintained at any time, the operation is convenient, the cleaning is timely, and the scale and sewage can be thoroughly cleaned, ensuring that the heater 406 is clean and hygiene and not fouled. Furthermore, the heating efficiency and heat conversion efficiency are enhanced, energy consumption is reduced, energy can be saved and environmental protection can be achieved.

The automatic opening and closing mechanism includes a turning motor, a flip motor, a turning sensor 701 and a flip sensor disposed in the toilet cover 300 and connected to the main control circuit board 800. The turning motor is used to drive the rotating shaft hinged with the seat ring 200 to rotate, thereby realizing the automatic flipping and closing operation, and the flip motor is used to drive the rotating shaft hinged with the toilet cover 300 to rotate, thereby realizing the automatic flipping and closing operation. The structure is simple and convenient to operate. In the present disclosure, the turning sensor 701 and the flip sensor are configured as a microwave sensor head or an infrared sensor head. This embodiment adopts an infrared sensor head. The infrared sensor head for controlling the turning and the flipping of the cover is disposed on the toilet cover 300. When the user performs a waving operation on the corresponding infrared sensor head, the infrared sensor head transmits the received gesture signal to the main control circuit board 800, and the main control circuit board 800 controls the turning motor and the flip motor to work, thereby realizing the automatic flipping and closing operation. The structure is simple and convenient to operate with high degree of automation and utility.

In this embodiment, the rear seat 102 is provided with a control panel connected to the main control circuit board 800. The control panel is provided with a plurality of control buttons and indicator lights, and the smart toilet can be controlled by the control buttons.

In summary, in the present disclosure, the smart toilet safe in use adopts steam to perform high-temperature sterilization and cleaning on the toilet seat 100 and the seat ring 200, thereby improving the cleanliness of the smart toilet, and effectively killing bacteria and cleaning the toilet, thereby preventing occurrence and propagation of diseases. Moreover, the main control circuit board 800 is connected to the radar sensor 500. When the steam sterilization button is pressed, the radar sensor 500 works and detects whether there is anyone moving within 6 meters. If human activity is detected, the main control circuit board 800 controls the steam generator 400 not to work according to the signal detected by the radar sensor 500, thereby preventing the generated high-temperature steam from burning the user's body. When the user's body leaves the detection range, the main control circuit board 800 controls the steam generator 400 to work and perform fumigation and sterilization on the toilet. If the steam generator 400 is working and performing fumigation and sterilization on the toilet while someone enters the sensing range of the radar sensor 500, the main control circuit board 800 controls the steam generator 400 to stop working according to the signal detected by the radar sensor 500 and to stop spraying steam, thereby effectively improving the safety performance in use and intelligence of the smart toilet. The structure is simple with reasonable design. In addition, the steam generator 400 can slide out of the toilet seat 100 from the inside of the toilet seat 100 through the slide rail mechanism 600 for ease of cleaning. The steam generator 400 is provided with a water inlet 401 connected to the water pump and a sewage outlet for cleaning the heater 406. The heater is brushed and cleaned by the sewage outlet, and the water pump is used to spray water and clean the sewage in the heater. The scale is discharged from the sewage outlet, the scale is removed timely, the heating efficiency and the heat conversion efficiency can be improved, and the steam generator 400 is kept clean and hygiene for a long time. The sanitary condition of the smart toilet is indirectly enhanced. In this manner, the present disclosure realizes a smart toilet which has a reasonable structural design and is clean and hygienic while having high safety performance in use and high heating efficiency, and has broad market prospects.

The above description is only a preferred embodiment of the present disclosure, and thus does not limit the scope of the present disclosure. Moreover, the equivalent structural modification made by using the descriptions and the contents of the drawings of the present disclosure as well as direct or indirect application of the present disclosure in other related technical field equally fall within the scope to be protected by the present disclosure.

What is claimed is:

1. A smart toilet safe in use, the smart toilet comprising a toilet seat, a rear seat, a seat ring hinged on the rear seat through a rotating shaft and a toilet cover, in the safe smart toilet further comprising a steam generating mechanism disposed in the toilet seat and spraying steam having fumigating and sterilizing function toward the toilet seat, an automatic opening and closing mechanism disposed in the rear seat and driving the seat ring and the toilet cover to open automatically, a main control circuit board disposed in the rear seat and controlling the steam generating mechanism and the automatic opening and closing mechanism to work, wherein the steam generating mechanism comprises a steam generator and a radar sensor connected to the main control circuit board; the steam generator is disposed in the toilet seat through a slide rail mechanism, and the toilet seat is provided with a cleaning hole corresponding in position to the slide rail mechanism and allowing the steam generator to slide out; the automatic opening and closing mechanism comprises a turning motor, a flip motor, a turning sensor and a flip sensor disposed in the toilet cover and connected to the main control circuit board.

2. The smart toilet according to claim 1, wherein the steam generator is provided with a water inlet connected to a water pump, a sewage outlet for cleaning a heater, and a steam outlet, the steam generator comprises a housing, a heat insulation shell, a heater cover, a heater, a temperature controller and a sewage outlet cover; the temperature controller is connected to the main control circuit board.

3. The smart toilet according to claim 1, wherein the slide rail mechanism comprises an inner slide rail fixed in the toilet seat, an outer slide rail fastened on the inner slide rail and sliding in a left-right direction along the inner slide rail, and the steam generator is fixed on the outer slide rail.

4. The safe smart toilet according to claim 2, wherein the slide rail mechanism comprises an inner slide rail fixed in the toilet seat, an outer slide rail fastened on the inner slide rail and sliding in a left-right direction along the inner slide rail, and the steam generator is fixed on the outer slide rail.

5. The smart toilet according to claim 3, wherein the toilet cover is provided with an inner cavity, and a lower end surface of the inner cavity is provided with a plurality of steam injecting holes at a position corresponding to a position of the seat ring, and the inner cavity communicates with the steam outlet through a conduit.

6. The smart toilet according to claim 5, wherein the turning sensor and the flip sensor are configured as a microwave sensor head or an infrared sensor head.

7. The smart toilet according to claim 6, wherein the rear seat is provided with a control panel connected to the main control circuit board, and the control panel is provided with a plurality of control buttons and indicator lights.

8. The smart toilet according to claim 7, wherein the cleaning hole is provided with a cleaning cover.
